(19) United States
(12) Patent Application Publication
(10) Pub. No.: US 2020/0319063 A1
(43) Pub. Date: Oct. 8, 2020

(54) SWITCH CABINET FOR EXHAUST-GAS MEASUREMENT INSTALLATIONS

(71) Applicant: AVL EMISSION TEST SYSTEMS GMBH, NEUSS (DE)

(72) Inventor: ACHIM DICKOW, VELBERT (DE)

(73) Assignee: AVL EMISSION TEST SYSTEMS GMBH, NEUSS (DE)

(21) Appl. No.: 16/305,058

(22) PCT Filed: Apr. 13, 2017
(86) PCT No.: PCT/EP2017/058929

§ 371 Date: Nov. 28, 2018

(30) Foreign Application Priority Data
May 31, 2016 (DE) 10 2016 110 063.3

(51) Int. Cl. G01N 1/22; G01D 11/30; G01N 33/00

(52) U.S. Cl. CPC G01N 1/2252; G01N 33/0027; G01D 11/305

(57) ABSTRACT

A switch cabinet for an exhaust gas measurement installation includes a cabinet body with a rear wall and four laterally bounding walls which include a ceiling, a floor, a first side wall and a second side wall. A door is arranged on the cabinet body to close a front side thereof. At least one valve, measuring devices, and gas connections are arranged in the cabinet body. The gas connections extend through the walls and interact with the at least one valve. The gas connections comprise at least one first gas connection arranged in a first horizontal plane and in a first vertical plane, and at least one second gas connection arranged in a second horizontal plane in a second vertical plane. The second horizontal plane is offset with respect to the first horizontal plane. The second vertical plane is offset with respect to the first vertical plane.

The invention relates to a switch cabinet for exhaust gas measurement installations having a cabinet body composed of four laterally bounding walls of which one serves as a ceiling, one serves as a floor and two serve as side walls, as well as a rear wall, and can be closed by a door arranged on the front side, wherein measuring devices and gas connections are arranged in the cabinet body, wherein the gas connections extend through the walls of the cabinet body and respectively interact with at least one valve arranged within the cabinet body.

Such switch cabinets are in particular used in the automotive industry for determining the emission behavior of motor vehicles. The motor vehicle performs a defined driving cycle on a roller-type test stand, wherein the emitted exhaust gases are collected, diluted, where necessary, and fed to a switch cabinet configured as an analysis unit. In the switch cabinet a power supply unit, a control unit, the electronic system as well as cooling units are arranged besides various measuring devices for detecting various components of the exhaust gas, such as carbon dioxide, hydrocarbon or nitrogen oxides, for example. Accordingly, the switch cabinet comprises a plurality of connections and lines via which measuring gas, zero gas and calibrating gas have to be delivered to the corresponding units. Moreover, such switch cabinets are used as gas distributor cabinets which essentially include pressure controllers and pressure sensors via which the system pressure is changed to the installation pressure.

A switch cabinet for an exhaust gas measurement installation is known from EP 2 317 833 A1, for example. This switch cabinet is composed of two cabinet parts. In the front cabinet part measuring devices as well as a computer are arranged, and in the rear cabinet part an inlet interface for introducing measuring gases, measuring gas pumps, valves and pressure reducers are arranged. The inlet interface is formed in the ceiling of the rear cabinet part and comprises many inlet lines arranged side by side and extending vertically in the rear portion of the switch cabinet. The measuring gas pumps, valves and pressure reducers are arranged upstream of the inlet lines, as seen from the open front side of the rear switch cabinet.

The configuration described in EP 2 317 833 A1 and generally such switch cabinets are problematic in that the switch cabinets are to be configured in a compact and space-saving manner, whereby, however, the components, such as the measuring devices, the valves and the gas connections, for example, arranged in the switch cabinet are not adequately accessible for facilitating in particular the maintenance of the switch cabinet.

It is therefore an object to enhance a switch cabinet for exhaust gas measurement installations in such a manner that the switch cabinet is of a space-saving configuration but nevertheless the components arranged in the switch cabinet, in particular the valves and the gas connections, as easily accessible.

This object is achieved by a switch cabinet for exhaust gas measurement installations with the features of the main claim 1.

Due to the fact that at least one first gas connection is arranged in a first horizontal plane and a first vertical plane, and at least one second gas connection is arranged in a second vertical plane offset to the first horizontal plane, the gas connections and the valves can be arranged in the switch cabinet in such a manner that the gas connections are easily accessible from outside and the valves are easily accessible from the open front side. Due to the horizontally and vertically offset arrangement of the second gas connection relative to the first gas connection no lines or other elements must be dismantled for accessing the valves and the gas connections of the respective other plane. Moreover, due to the offset arrangement of the first gas connection relative to the second gas connection the installation space of the switch cabinet can be reduced.

Preferably, the at least one first gas connection is arranged nearer to the door than the at least one second gas connection.

Preferably, the at least one first gas connection and the at least one second gas connection are vertically oriented and extend through the ceiling of the switch cabinet, wherein the at least one first gas connection is arranged further to the outside from the central horizontal plane of the switch cabinet than the at least one second gas connection. Alternatively, the at least one first gas connection and the at least one second gas connection are horizontally oriented and extend through a side wall of the switch cabinet, wherein the at least one first gas connection is arranged further to the outside from the central vertical plane of the switch cabinet than the at least one second gas connection. Due to such an arrangement of the gas connections, the valves of the gas connections, in particular the valves interacting with the second gas connection, are easily accessible through the open front side since no lines have to be guided along the second connection strip.

Preferably, the ceiling or one of the side walls are configured in a stepped manner with a first step and a second step, wherein at the first step the at least one first connection is arranged and at the second step the at least one second connection is arranged. This results, in a simple and inexpensive manner, in the arrangement of the gas connections horizontally and vertically offset to each other.

According to a preferred aspect, a removable ceiling element is arranged between the two steps. The removable ceiling element closes the switch cabinet towards the environment and, by simply removing the ceiling element, allows for a better accessibility of the second gas connection.

Preferably, the at least one first and the at least one second gas connection are calibrating gas, zero gas or measuring gas connections. Thus all gas connections required for the exhaust gas measurement can be arranged such that the switch cabinet can be configured in a space-saving manner and all gas connections and the valves are easily accessible.

According to an advantageous aspect, the at least one first gas connection and the at least one second gas connection are fastened via a respective adapter element fixed at the cabinet body, wherein the adapter element comprises at least one inlet opening and at least one outlet opening which are connectable to each other via the at least one valve arranged at the adapter element. Thus the gas connection and the associated valve are arranged in the cabinet body in a simple and space-saving manner. Moreover, the mounting effort and the number of components are reduced.

Preferably, several adapter elements can be assembled and the gas ducts formed in the adapter elements are fluidically connectable to each other, whereby a calibrating unit can be provided which, depending on the required gas connections, can be made larger or smaller and can be preassembled in a simple manner.

According to an advantageous embodiment, several adapter elements are connected to a first connection strip and to a second connection strip, wherein the first connection strip comprises a plurality of first gas connections and the second connection strip comprises a plurality of second gas connections. Thus several adapter elements can be preassembled to form a unit before being mounted into the switch cabinet, whereby mounting into the switch cabinet is facilitated and several persons are allowed to work at the switch cabinet.

Preferably, the first connection strip and the second connection strip are respectively fastened to the cabinet body via a fastening plate, whereby the connection strips can be fastened in the switch cabinet in a completely preassembled manner.

According to a preferred aspect, in the switch cabinet at least one third gas connection is arranged which is vertically oriented to the at least one first gas connection and the at least one second gas connection. Thus three gas connections can be arranged in the switch cabinet in a space-saving manner by arranging two gas connections at the ceiling and one gas connection at a side wall, for example.

Preferably, at each fastening element at least one circuit board is arranged which is electrically connected to the at least one valve. According to a particularly preferred aspect, at the fastening elements a first and a second circuit board are respectively arranged which are disposed such that they are horizontally and vertically offset to each other. Thus the circuit board can be arranged in the cabinet body in a space-saving manner in the immediate vicinity of the valves such that the length of the electric lines is reduced.

There is thus provided a switch cabinet for exhaust gas measurement installations which allows for a space-saving arrangement of the gas connections while the gas connections as well as the valves associated with the gas connections are easily accessible, whereby the maintenance and the mounting of the switch cabinet are facilitated.

An exemplary embodiment of a switch cabinet according to the invention for an exhaust gas measurement installation is illustrated in the Figures and is described below.

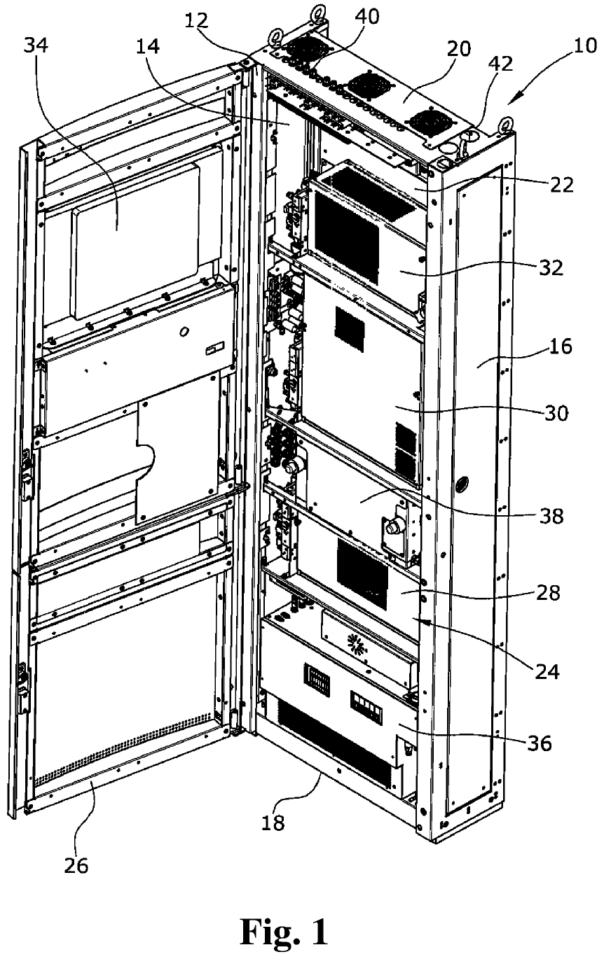

FIG. 1 shows a front view of a switch cabinet for an exhaust gas measurement installation with an arrangement of the gas connections according to the invention.

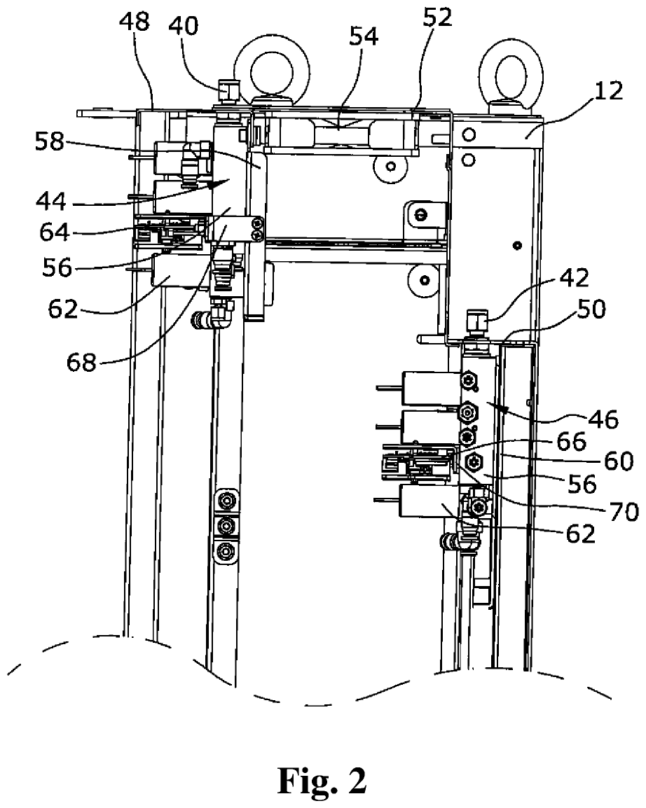

FIG. 2 shows a cross-sectional side view of the switch cabinet of FIG. 1 with an arrangement of the gas connections according to the invention.

The switch cabinet 10 for an exhaust gas measurement installation is composed of a cabinet body 12 having four laterally bounding walls 14, 16, 18, 20, in particular a first side wall 14, an opposed second side wall 16, a floor 18 and a ceiling 20, as well as a rear wall 22. An open front side 24 of the cabinet body 12 can be closed by means of a door 26 which is pivotably fastened to the first side wall 14.

In the switch cabinet 10, measuring devices 28, 30, 32 for analyzing measuring gases are arranged which are connected to measuring gas connections arranged at the side wall 16 and not shown in the Figures and which are controllable via an operating unit 34 arranged in the door 26.

In the cabinet body 12, all components required for analysis of a measuring gas are arranged. On the floor 18 of the cabinet body 12 a power supply unit 36 is arranged via which the measuring devices 28, 30, 32 and other components arranged in the cabinet body 12 are supplied with power. Above the power supply unit 36 the first measuring device 28 is arranged which is an infrared-detector analyzing device and is used for detecting carbon monoxide, carbon dioxide or hydrocarbon compounds, for example. The measuring gas in the infrared-detector analyzing device 28 is cooled via a cooler 38 arranged above the infrared-detector analyzing device 28 and freed from condensate.

Above the cooler 38, the second measuring device 30 is arranged which is configured as a chemiluminescence analyzing device via which the amount of nitrogen oxide in the exhaust gas is measured.

Again above the chemiluminescence analyzing device 30, a flame-detector ionization analyzing device for detecting hydrocarbons is arranged as a third measuring device 32.

At the ceiling 20 of the cabinet body 12, gas connections 40, 42 for calibration as well as for zero balancing of the measuring devices 28, 30, 32 are arranged. Via the gas connections 40, 42, zero and calibrating gases are introduced into the switch cabinet 10 and discharged therefrom, respectively. In this configuration, the gas connections for introducing measuring gases are arranged at the second side wall 16 and not shown in the Figures. In other configurations of the switch cabinet 10, the measuring gas connections can be configured in the same manner as the gas connections 40, 42 and arranged at the ceiling 20.

FIG. 2 shows an arrangement of the gas connections 40, 42 at the ceiling 20 of the cabinet body 12 according to the invention. A plurality of first gas connections 40 are connected to a first connection strip 44, and a plurality of second gas connections 42 are connected to a second connection strip 46, wherein the first connection strip 44 is arranged such that it is horizontally and vertically offset to the connection strip 46 in the cabinet body 12. For such an offset arrangement of the connection strips 44, 46, the ceiling 20 comprises two steps 48, 50. The first step 48 adjoins the front side 24 of the cabinet body 12 and is arranged further to the outside than the second step 50, as seen from the central horizontal plane of the cabinet body 12. The second step 50 adjoins the rear side 22 of the cabinet body 12. Thus at the first step 48 the first connection strip 44 is arranged and at the second step 50 the second connection strip 46 is arranged. Between the two steps 48, 50, another easy-to-remove ceiling element 52 is arranged which serves as a carrier for a fan 54 and is configured as an extension of the first step 48 in the mounted condition.

The connection strips 44, 46 are each made up of several assembled adapter elements 56 and are fastened to the rear wall 22 of the cabinet body 12 and the vertically extending wall of the first step 48, respectively, via a respective fastening element 58, 60. Each adapter element 56 comprises an opening which has an internal thread and faces the ceiling 20, into which opening the first and the second gas connection 40, 42 configured as inlet stubs are screwed.

A gas duct formed in the adapter element 56 and not shown in the Figures adjoins the gas connections 40, 42 screwed into the adapter element 56, which duct extends from the gas connections 40, 42 to an outlet opening also not shown. From the outlet opening, the zero and calibrating gases, respectively, flow to the corresponding measuring devices. The gas mass flow through the gas connections 40, 42 is released or stopped by a respective valve 62 arranged at the adapter element 56 and controlled, where necessary.

The adapter element 56 comprises additional gas ducts via which different gas connections 40, 42 of the respective connection strip 44, 46 are connected to each other.

At the lower end of the connection strips 44, 46 a respective circuit board 64, 66 is arranged to which the valves 62 are electrically connected. The circuit boards 64, 66 are fastened to the fastening element 58, 60 via a respective carrier plate 68, 70.

During the maintenance of the switch cabinet 10, the gas connections 40,42 and the associated valves 62 must be easy to access. The switch cabinet 10, whose rear wall 22 is placed at a building wall, should not be displaced. The first gas connections 40 of the first connection strip 44 are easily accessible for the maintenance personnel from the front side 24 since the first gas connections 40 are directly arranged at the end of the ceiling 20 facing the front side 24. The second gas connections 42 are also accessible from outside. For a better access to the second gas connections 42 of the second connection strip 46 without displacing the switch cabinet 10, the ceiling element 52 with the fan 54 fastened thereto can be removed, whereby the second gas connections 42 are accessible from the top or from the front. The valves 62 of the two connection strips 44, 46, which are arranged in the cabinet body 12 such that they are offset to one another, as well as the screw connection of the gas connections 42 are easily accessible via the front side 24 when the door 26 is open since no lines have to be guided along the second connection strip 46.

Thus a switch cabinet for exhaust gas measurement installations is provided which allows for a space-saving arrangement of the gas connections while the gas connections as well as the valves associated with the gas connections are easily accessible and thus the maintenance of the switch cabinet is facilitated.

It should be appreciated that the scope of protection of the present main claim is not limited to the described exemplary embodiment. In particular, the switch cabinet can be equipped with other or additional measuring devices. Furthermore, the gas connections can be arranged at one of the side walls or on the floor of the switch cabinet such that they are offset to each other.

1-14. (canceled)

15. A switch cabinet for an exhaust gas measurement installation, the switch cabinet comprising:
a cabinet body comprising a rear wall and walls arranged to be laterally bounding, the walls comprising a ceiling, a floor, a first side wall, and a second side wall;
a door arranged on the cabinet body to close a front side of the cabinet body;
at least one valve arranged in the cabinet body;
measuring devices arranged in the cabinet body; and
gas connections arranged in the cabinet body, the gas connections being configured to extend through the walls of the cabinet body and to respectively interact with the at least one valve arranged in the cabinet body, the gas connections comprising,
at least one first gas connection which is arranged in a first horizontal plane and in a first vertical plane, and
at least one second gas connection which is arranged in a second horizontal plane in a second vertical plane,
wherein,
the second horizontal plane is offset with respect to the first horizontal plane, and
the second vertical plane is offset with respect to the first vertical plane.

16. The switch cabinet as recited in claim 15, wherein the at least one first gas connection is arranged closer to the door than the at least one second gas connection.

17. The switch cabinet as recited in claim 15, wherein,
the cabinet body further comprises a central horizontal plane,
the at least one first gas connection and the at least one second gas connection are each oriented vertically to extend through the ceiling of the switch cabinet, and
the at least one first gas connection is arranged further to an outside of the central horizontal plane of the cabinet body than is the at least one second gas connection.

18. The switch cabinet as recited in claim 17, wherein,
the ceiling is configured in a stepped manner comprising a first step and a second step,
the at least one first gas connection is arranged at the first step, and
the at least one second gas connection is arranged at the second step.

19. The switch cabinet as recited in claim 18, wherein the cabinet body further comprises a removable ceiling element which is arranged between the first step and the second step.

20. The switch cabinet as recited in claim 15, wherein,
the cabinet body further comprises a central vertical plane,
the at least one first gas connection and the at least one second gas connection are each oriented horizontally to extend through at least one of the first side wall and the second side wall of the cabinet body, and
the at least one first gas connection is arranged further to an outside of the central vertical plane of the cabinet body than is the at least one second gas connection.

21. The switch cabinet as recited in claim 20, wherein,
at least one of the first side wall and the second side wall are configured in a stepped manner with a first step and a second step,
the at least one first gas connection is arranged at the first step, and
the at least one second gas connection is arranged at the second step.

22. The switch cabinet as recited in claim 15, wherein the at least one first gas connection and the at least one second gas connection are a calibrating gas connection, a zero gas connection, or a measuring gas connection.

23. The switch cabinet as recited in claim 15, further comprising:
adapter elements fastened at the cabinet body, each of the adapter elements comprising at least one inlet opening and at least one outlet opening which are connectable to each other via the at least one valve which is further arranged at each of the adapter elements,
wherein,
the at least one first gas connection and the at least one second gas connection are each connected to a respective one of the adapter elements.

24. The switch cabinet as recited in claim 23, wherein,
the adapter elements are each configured to have a gas duct be formed therein, and
the adapter elements are fluidically connectable to each other.

25. The switch cabinet as recited in claim 23, further comprising:
a first connection strip comprising a plurality of the at least one first gas connections, and
a second connection strip comprising a plurality of the at least one second gas connections,
wherein,
a plurality of the adapter elements are connected to the first connection strip and to the second connection strip.

26. The switch cabinet as recited in claim 25, further comprising:
fastening elements,
wherein,
the first connection strip is fastened to the cabinet body via one of the fastening elements, and
the second connection strip is fastened to the cabinet body via one of the fastening elements.

27. The switch cabinet as recited in claim 26, further comprising:
circuit boards,
wherein,
one of the circuit boards is arranged at each of the fastening elements, and
each of the circuit boards is electrically connected to the at least one valve.

28. The switch cabinet as recited in claim 27, wherein,
the circuit boards comprise a first circuit board and a second circuit board, and
the first circuit board and the second circuit board are arranged at a respective one of the fastening elements so as to be horizontally and vertically offset with respect to each other.

29. The switch cabinet as recited in claim 15, further comprising:
at least one third gas connection arranged in the cabinet body, the at least one third gas connection being oriented vertically to the at least one first gas connection and to the at least one second gas connection.
